SEQUENCE LISTING (22 sequences)

SEQ ID NO: 1 - DNA, 21 bp, Artificial Sequence
  primer
gccttcaactgcaaatactgc

SEQ ID NO: 2 - DNA, 20 bp, Artificial Sequence
  primer
tgacatctgagtgggtctgg

SEQ ID NO: 3 - DNA, 20 bp, Artificial Sequence
  primer
ttcggacccacacattacct

SEQ ID NO: 4 - DNA, 20 bp, Artificial Sequence
  primer
ttggagcagtttttgcactg

SEQ ID NO: 5 - DNA, 20 bp, Artificial Sequence
  primer
ggatttgccttgtctgaagc

SEQ ID NO: 6 - DNA, 20 bp, Artificial Sequence
  primer
cgtacattcccatgacacca

SEQ ID NO: 7 - DNA, 20 bp, Artificial Sequence
  primer
gaagtggagaaagcctgtgc

SEQ ID NO: 8 - DNA, 20 bp, Artificial Sequence
  primer
ctctgtgagcagtgccaaaa

SEQ ID NO: 9 - DNA, 20 bp, Artificial Sequence
  primer
ggggtgttgaaggtctcaaa

SEQ ID NO: 10 - DNA, 20 bp, Artificial Sequence
  primer
ggcatcctcaccctgaagta

SEQ ID NO: 11 - DNA, 20 bp, Artificial Sequence
  primer
accgtgttcttcgacattgc

SEQ ID NO: 12 - DNA, 20 bp, Artificial Sequence
  primer
gcctccacaatattcatgcc

SEQ ID NO: 13 - DNA, 20 bp, Artificial Sequence
  primer
ggagaatcctgggggttatg

SEQ ID NO: 14 - DNA, 20 bp, Artificial Sequence
  primer
gctttcagcatttgcaacct

SEQ ID NO: 15 - DNA, 21 bp, Artificial Sequence
  primer
gagacaacctggtcctctgtg

SEQ ID NO: 16 - DNA, 20 bp, Artificial Sequence
  primer
cttggctggtttctccagac

SEQ ID NO: 17 - DNA, 20 bp, Artificial Sequence
  primer
cattgagggtctgatgagca

SEQ ID NO: 18 - DNA, 20 bp, Artificial Sequence
  primer
aggtccaccactgagacgtt

SEQ ID NO: 19 - DNA, 20 bp, Artificial Sequence
  primer
gactgagaagctgggtttgg

SEQ ID NO: 20 - DNA, 20 bp, Artificial Sequence
  primer
tggtaccaccagacagcact

SEQ ID NO: 21 - DNA, 20 bp, Artificial Sequence
  primer
ggcttctctctccaccttcc

SEQ ID NO: 22 - DNA, 20 bp, Artificial Sequence
  primer
gactgagaagctgggtttgg

(19) United States
(12) Patent Application Publication
(10) Pub. No.: US 2015/0252413 A1
(43) Pub. Date: Sep. 10, 2015

(54) METHOD FOR NORMALIZATION OF QUANTITATIVE PCR AND MICROARRAYS

(71) Applicant: BIOQUANTA, Paris (FR)

(72) Inventors: Marc Conti, Palaiseau (FR); Sylvain Loric, Brunoy (FR); Phillippe Manivet, Paris (FR); Carlosse Keumeugni, Villeneuve La Garenne (FR); Deborah Revaud, Mondeville (FR); Jean-Luc Gala, Zaventem (BE)

(21) Appl. No.: 14/356,789

(22) PCT Filed: Nov. 7, 2012
(86) PCT No.: PCT/EP2012/072062

§ 371 Date: May 7, 2014

Related U.S. Application Data
(60) Provisional application No. 61/556,655, filed on Nov. 7, 2011.

(51) Int. Cl. C12Q 1/68; G06F 19/20

(52) U.S. Cl. CPC C12Q 1/6851; C12Q 1/6837; G06F 19/20

(57) ABSTRACT

The present invention concerns a method for comparing, in at least two samples A₁ and A₂, the amount of RNA of a target gene t, wherein a fixed amount of external control sample C is added during RNA extraction of samples A₁ and A₂ and a fixed amount of external control D RNA is added to the extracted RNA from A₁+C and A₂+C before reverse transcription.

The present invention concerns methods for normalizing the results obtained from quantitative PCR or microarrays.

Gene expression analysis is one of the most interesting ways to compare experimental or clinical conditions. Understanding gene expression profiles is expected to provide insight into complex regulatory networks. Over the last twenty years, real time quantitative PCR (rt-qPCR) has become the method of choice for accurate expression profiling, replacing end-point PCR, RPA (Ribonuclease Protection Assay) and Northern blotting. Although this method is widely used, much needs to be done to increase its reliability and accuracy.

Typically, rt-qPCR requires different steps (FIG. 1):
Extraction of RNA from sample (Step 1)
Reverse Transcription (RT) to produce cDNA (Step 2)
Amplification and real time quantification of cDNA template (rt-qPCR) (Step 3)
Detection and analysis (Step 4)

Each of these steps has a variable yield that could alter quantification of the target gene. In addition, PCR suffers from false negative results when enzyme inhibitors are present in the samples or when reagents are missing or degraded.

Steps 1 and 2 are also used for microarray experiments. Accordingly, controls applicable to these steps are relevant for these techniques (FIG. 1).

To ensure normalization of initial steps of rt-qPCR and microarrays, different improvements have been developed (van de Peppel et al. (2003) EMBO Rep. 4:387-393; Huggett et al. (2005) Genes Immun 6:279-284). These improvements are summarized in Table 1.

TABLE 1
Comparison of the different normalization strategies used for RT-PCR (from
Huggett et al. (2005)).

| Normalisation | | | |
|---|---|---|---|
| strategy | Pros | Cons | Note |
| Similar sample | Relatively easy | Sample size/tissue | Simple first step to |
| size/tissue volume | | volume may be difficult to | reduce experimental |
| | | estimate and/or may not | error |
| | | be biological | |
| | | representative | |
| Total RNA | Ensures similar reverse | Does not control for error | Requires a good |
| | transcriptase input. May | introduced at the reverse | method of assessing |
| | provide information on | transcription or PCR | quality and quantity |
| | the integrity (depending | stages. Assumes no | |
| | on technique used) | variation in rRNA/mRNA | |
| | | ratio | |
| Genomic DNA | Gives an idea of the | May vary in copy number | Rarely used. Can be |
| | cellular sample size. | per cell. Difficult to | measured optically or |
| | | extract with RNA | by real time PCR |
| Reference genes | Internal control that is | Must be validated using | Oligo dt priming of |
| ribosomal RNAs | subject to the same | the same experimental | RNA for reverse |
| (rRNA) | conditions as the RNA | samples Resolution of | transcription will not |
| | of interest. | assay is defined by the | work well with rRNA |
| | | error of the reference | as no polyA tail is |
| | | gene | present. Usually in |
| | | Ribosomal RNAs are not | high abundance |
| | | transcribed as | |
| | | messenger RNAs | |
| Reference genes | Internal control that is | Must be validated using | Most, but not all, of |
| messenger RNAs | subject to the same | the same experimental | mRNAs contain |
| (mRNA) | conditions as the | samples. Resolution of | polyA tails and can |
| | mRNA of interest. | assay is defined by the | be primed with oligo |
| | | error of the reference | dt for reverse |
| | | gene | transcription |
| Alien molecules | Internal control that is | Must be identified and | Requires more |
| | subject to most of the | cloned or synthesized. | characterization and |
| | conditions as the | Unlike the RNA of | development to be |
| | mRNA of interest. Is | interest, is not extracted | as similar as possible |
| | without the biological | from the within the cells | that natural RNA |
| | variability of a reference | | molecule |
| | gene | | |

One way to normalize target mRNA expression is to use a fixed amount of total RNA for subsequent RT, namely “total RNA normalization”. Total RNA normalization is deemed inaccurate because total RNA is mainly composed of ribosomal RNA (rRNA) which amount is too different from the amount of the messenger RNA (mRNA) of interest. Furthermore, total RNA normalization does not control for RNA degradation or output variations during quantification of RNA molecules or RT.

The most commonly used way to normalize gene expression is to report the expression of the gene(s) of interest to the expression of “HouseKeeping Genes” (HKG) or internal control genes which expression is assumed to be stable between cells/tissues/samples and experimental conditions. These HKG can code for mRNA or rRNA. Nevertheless, the use of rRNA is not a good standard because these RNAs are present in the cell in a much larger quantity than the target mRNA. In addition, the present inventors (Caradec et al. (2010) Br. J. Cancer 102:1037-1043) and others (Lee et al. (2002) Genome Res. 12:292-297; Vandesompele et al. (2002) Genome Biol. 3:RESEARCH0034; Radonic et al. (2004) Biochem. Biophys. Res. Commun. 313:856-862) have demonstrated that HKG expression could vary according to samples or experimental procedures, leading to an inaccurate normalization, a misinterpretation of results and even conflicting report.

Most microarray experiments make use of the expression levels of all genes as normalization features, assuming that relatively few transcript levels vary between samples, or that any changes that occur are balanced. Van de Peppel et al. showed that this “all-gene” approach does not take into account global changes that often occur during experimental conditions, sampling and sample preparation (van de Peppel et al. (2003)).

Therefore there are no universal control genes to normalize rt-qPCR and microarray assays. Accordingly, prior to any quantification of target genes, several HKG should be tested for their stability in each condition or sample studied (pre-experimental validation) in order to determine the less variable HKG which will be the more appropriate reference for the experiment (Tricarico et al. (2002) Anal Biochem 309:293-300; Pfaffl et al. (2004) Biotechnol Lett 26:509-515). This prior analysis is cumbersome, time-consuming and costly. Furthermore, for laboratories which are not using rt-qPCR or microarray assay as a routine, the prior study of HKG is not profitable. Finally, searching for the best HKG means the pre-analysis consumption of precious samples.

To determine the best HKG among set of reference genes tested, mathematical models (Chervoneva et al. (2010) BMC Bioinformatics 11:253) and specialized softwares (Vandesompele et al. (2002); Pfaffl et al. (2004)) have been developed. However, mathematical models can be complex and difficult to use and specialized softwares do not always corroborate on the determination of the best HKG. In addition, different primers are used for common HKG amplification, which are not always the same between laboratories. The sequences of primers could influence rt-qPCR efficiency resulting in variation of results obtained using the same HKG in the same experimental protocol in different laboratories. Therefore, there are neither universal control genes nor universal defined protocols to determine the best control genes.

Nowadays, the use of HKG in the normalization of rt-qPCR and microarray results does not seem an accurate way for a universal standardization of results and worldwide comparison of genomic expression. Scientific community suggested developing universal RNA material reference for rt-qPCR and microarrays standardization. The addition of external heterologous RNA (either synthetic as alien RNA or from plants when studying animal genes for example) at the RT step (Step 2) is considered the most promising method to normalize results. It allows monitoring of the RT, PCR efficiency and RNA degradation.

However, it is of no help to monitor the variation of extraction yield, errors in total RNA quantification or the degradation of the RNA of interest during storage. In addition, since synthetic RNAs are in vitro retro-transcribed, concerns may arise on the different efficiencies of RT and PCR compared to natural RNA with secondary and tertiary structures. Exogenous RNAs are already used for microarrays (Benes and Muckenthaler (2003) Trends Biochem Sci 28:244-249) but their use for normalization of rt-qPCR results is not generalized (Huggett et al. (2005) Genes Immun 6:279-284).

To conclude, there is a real need for an easy-to-use, commercially available method of normalization to monitor all the pre-analytical steps of gene expression analysis assays such as rt-qPCR and microarrays and to facilitate the comparison of data collected in laboratories throughout the world. Reliable normalization methods are also mandatory for microarray and rt-qPCR methods to be widely adopted for clinical diagnostic use.

The present invention arises from the unexpected finding by the inventors that addition of a fixed amount of external control sample during extraction of RNAs to be studied and of a fixed amount of external RNA before the RT, as depicted in FIG. 2, enables normalizing easily extraction of RNA, reverse transcription and the consecutive processes of qPCR and microarrays.

The present invention thus concerns a method for comparing, in at least two samples A₁ and A₂, the amount of RNA of a target gene t, comprising the steps consisting of:
a) mixing each of the at least two samples A₁ and A₂ with a determined amount of external control sample C comprising RNA of a reference gene g_{c};
b) extracting RNA from each of the at least two mixtures A₁+C and A₂+C obtained in step a), in order to obtain corresponding solutions of extracted RNA;
c) mixing each of the at least two solutions of extracted RNA of A₁+C and A₂+C with a determined amount of external control D RNA including RNA of a reference gene g_{d};
d) performing reverse transcriptions on each of the at least two mixtures A₁+C+D and A₂+C+D obtained in step c), in order to obtain corresponding solutions comprising cDNAs of the target gene t, of the reference gene g_{c} and of the reference gene g_{d};
e) measuring the cDNA levels of each of the target gene t, of the reference gene g_{c} and of the reference gene g_{d} in each of the at least two cDNA solutions A₁+C+D and A₂+C+D obtained in step d); and
f) normalizing the cDNA levels of the target gene t from the at least two samples A₁ and A₂, using cDNA levels of the reference genes g_{c} and g_{d};

wherein the reference gene g_{c} is selected in such a way that nucleic acids, primers and/or probes used in step e) to measure the cDNA level of the reference gene g_{c} do not cross-react with cDNAs of the target gene t and of the reference gene g_{d} and wherein the reference gene g_{d} is selected in such a way that nucleic acids, primers and/or probes used in step e) to measure the cDNA level of the reference gene g_{d} do not cross-react with cDNAs of the target gene t and of the reference gene g_{c}.

The present invention also concerns a kit for comparing the amount of RNA of a target gene t in at least two samples A₁ and A₂, comprising:
(i) a determined amount of an external control sample C comprising RNA of a reference gene g_{c} and of a reference gene g′_{c};
(ii) a determined amount of external control D RNA including RNA of a reference gene g_{d} and of a reference gene g′_{d};
(iii) a couple of primers that specifically amplify cDNA of the reference gene g_{c};
(iv) a couple of primers that specifically amplify cDNA of the reference gene g′_{c};
(v) a couple of primers that specifically amplify cDNA of the reference gene g_{d}; and
(vi) a couple of primers that specifically amplify cDNA of the reference gene g′_{d};

wherein the reference genes g_{c} and g_{d} are genes with a relative low expression level and the reference genes g′_{c} and g′_{d} are genes with a relative high expression level.

### DETAILED DESCRIPTION OF THE INVENTION

### Definitions

In the context of the invention, the terms “target gene”, “target RNA” and “target cDNA” refer to the sequences of interest to be quantified and/or compared in samples A₁ and A₂.

As used herein, the term “sample” refers to any biological or synthetic sample containing ribonucleic acid which can be extracted. Preferably, the samples of the invention are biological samples. In particular, the biological samples may be selected from the group consisting of blood, serum, plasma, urine, feces, cerebrospinal fluid, sperm, puncture fluid, expectora, saliva, bronchial and alveolar fluids, pus, genital secretions, amniotic fluids, gastric fluids, bile, pancreatic fluid, tissue biopsy, hair, skin, teeth, and lymphatic fluids. In the context of the invention, the biological sample can also constituted of cultured cells or medium containing ribonucleic acid. In some embodiments, biological samples may be synthetic and/or man-made, or a mix of natural and synthetic and/or man-made samples. In other embodiments, biological samples may be of beverages, perfumes, foods, or any type of fluids that could contain ribonucleic acids.

As used herein, the expression “external control sample” refers to a sample as defined above, preferably a biological sample as defined above, which is obtained from a different organism from the biological samples to be studied and which comprises RNA of a reference gene g_{c} as defined herein below.

As used herein, the expression “external control RNA” refers to a composition consisting essentially of RNA and which comprises RNA of a reference gene g_{d} as defined herein below.

As used herein, the expression “reference gene” refers to a gene, the sequence of which is used for normalization, and which relative level of expression in the external control biological sample or in the external control RNA is known. In the context of the invention, the reference gene g_{c} is selected in such a way that nucleic acids, primers and/or probes used in step e) of the method of the invention to measure the cDNA level of the reference gene g_{c} do not cross-react with cDNAs of the target gene t and of the reference gene g_{d} and the reference gene g_{d} is selected in such a way that nucleic acids, primers and/or probes used in step e) of the method of the invention to measure the cDNA level of the reference gene g_{d} do not cross-react with cDNAs of the target gene t and of the reference gene g_{c}.

As used herein, the term “cross-reacting” means hybridizing to and/or amplifying another nucleic acid sequence than the nucleic acid of interest.

In particular, the reference genes g_{c} and g_{d} may be homologous genes obtained from distinct species.

Preferably, the reference genes g_{c} and g_{d} are selected in such a way that the order of magnitude of their relative expression level is similar to the order of magnitude of the relative expression level of the target gene t. In particular, when the relative expression level of the target gene t is low, the reference genes g_{c} and g_{d} preferably display a low relative expression level. Similarly, when the relative expression level of the target gene t is high, the reference genes g_{c} and g_{d} preferably display a high relative expression level. Reference genes g_{c} and g_{d} displaying a low relative expression level or a high relative expression level are well-known from the skilled person or can be easily determined by the skilled person using conventional techniques of measurement of RNA level in a sample.

### Method of Comparison

The present invention concerns a method for comparing, in at least two samples A₁ and A₂, preferably at least two biological samples A₁ and A₂, the amount of RNA of a target gene t, comprising the steps consisting of:

a) mixing each of the at least two samples A₁ and A₂ with a determined amount of external control sample C, preferably of external control biological sample C, comprising RNA of a reference gene g_{c};

b) extracting RNA from each of the at least two mixtures A₁+C and A₂+C obtained in step a), in order to obtain corresponding solutions of extracted RNA;

c) mixing each of the at least two solutions of extracted RNA of A₁+C and A₂+C with a determined amount of external control D RNA including RNA of a reference gene g_{d};

d) performing reverse transcriptions on each of the at least two mixtures A₁+C+D and A₂+C+D obtained in step c), in order to obtain corresponding solutions comprising cDNAs of the target gene t, of the reference gene g_{c} and of the reference gene g_{d};

e) measuring the cDNA levels of each of the target gene t, of the reference gene g_{c} and of the reference gene g_{d} in each of the at least two cDNA solutions A₁+C+D and A₂+C+D obtained in step d); and

f) normalizing the cDNA levels of the target gene t from the at least two samples A₁ and A₂, using cDNA levels of the reference genes g_{c} and g_{d};

wherein the reference gene g_{c} is selected in such a way that nucleic acids, primers and/or probes used in step e) to measure the cDNA level of the reference gene g_{c} do not cross-react with cDNAs of the target gene t and of the reference gene g_{d} and wherein the reference gene g_{d} is selected in such a way that nucleic acids, primers and/or probes used in step e) to measure the cDNA level of the reference gene g_{d} do not cross-react with cDNAs of the target gene t and of the reference gene g_{c}.

RNA can be extracted from the sample according to any method well known to those of skill in the art. For example, methods of extraction of nucleic acids are described in Laboratory Techniques in Biochemistry and Molecular Biology: Hybridization With Nucleic Acid Probes, Part I. Theory and Nucleic Acid Preparation, P. Tijssen, ed. Elsevier (1993).

The extracted RNAs can be labeled with one or more labeling moieties to allow for detection of hybridized arrayed/sample nucleic acid molecule complexes. The labeling moieties can include compositions that can be detected by spectroscopic, photochemical, biochemical, bioelectronic, immunochemical, electrical, optical or chemical means. The labeling moieties include radioisotopes, such as ³²P, ³³P or ³⁵S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like. Preferred fluorescent markers include Cy3 and Cy5 fluorophores (Amersham Pharmacia Biotech, Piscataway N.J.).

As used herein, the term “reverse transcription” refers to the transcription of single-stranded RNA into single-stranded DNA (cDNA). Techniques to perform reverse-transcription are well-known from the skilled person and typically involve the use of reverse transcriptases. Preferably, the step of reverse transcription is performed by RT-PCR, i.e. techniques applying a polymerase chain reaction (PCR) after conversion of RNA into complementary DNA (cDNA) by a reverse transcription.

Techniques to measure cDNA levels in step e) are well-known from the skilled person. Such techniques include in particular quantitative PCR and microarrays.

As used herein, the expressions “quantitative PCR”, “real time PCR” and “real time RT-PCR” are used indifferently and refer to fluorescence-based PCR methods on photometric thermocyclers with the option for quantification of original template amounts. These techniques can include additional pre-amplification steps on a traditional thermocycler for a defined number of PCR-cycles.

Preferably, the step e) of measuring cDNA levels is performed by quantitative PCR.

Preferably, when step e) of measuring cDNA levels is performed by quantitative PCR, a cycle threshold Ct value is preferably obtained in step e) for target gene t, and for reference genes g_{c} and g_{d} in each of the at least two cDNA solutions A₁+C+D and A₂+C+D.

As used herein, the term “Cycle Threshold” or “Ct” refers to the cycle in exponential phase where fluorescent intensity reaches a predetermined manual or computed threshold level, significantly higher than the fluorescent background noise. Ct value thus logarithmically depends on the amount of template cDNA input and characterizes gene expression level.

As used herein, the term “normalizing” or “normalization” refers to a process enabling, when comparing the expression level of a gene in two samples, cancelling the differences due to the variable yields of each step of the comparison method.

In the context of the invention, in particular when step e) of measuring cDNA levels is performed by quantitative PCR and when a cycle threshold Ct value is obtained in step e) for target gene t, and for reference genes g_{c} and g_{d}, any calculation using the Ct values obtained for reference genes g_{c} and g_{d} can be used to normalize the results and give an estimation of experiment's yield between samples A₁ and A₂.

Preferably, in the method of the invention, step f) of normalizing is performed using the following equation:

$$R = \frac{{2}^{- [ Ct ( {t}^{{A}_{1}} ) - Ct ( {g}_{c}^{{A}_{1}} ) ] + Ct ( {g}_{d}^{{A}_{2}} ) - Ct ( {g}_{d}^{{A}_{1}} )}}{{2}^{- [ Ct ( {t}^{{A}_{2}} ) - Ct ( {g}_{c}^{{A}_{2}} ) ]}} ;$$

wherein:

R represents the ratio of the cDNA level of the target gene t in the sample A₁ on the cDNA level of the target gene t in the sample A₂,
Ct(t^{A}^{₁}) represents the cycle threshold obtained for the target gene t in the mixture A₁+C+D;
Ct(t^{A}^{₂}) represents the cycle threshold obtained for the target gene t in the mixture A₂+C+D;
Ct(g_{c}^{A}^{₁}) represents the cycle threshold obtained for the reference gene g_{c} in the mixture A₁+C+D;
Ct(g_{c}^{A}^{₂}) represents the cycle threshold obtained for the reference gene g_{c} in mixture A₂+C+D;
Ct(g_{d}^{A}^{₁}) represents the cycle threshold obtained for the reference gene g_{d} in the mixture A₁+C+D; and
Ct(g_{d}^{A}^{₂}) represents the cycle threshold obtained for the reference gene g_{d} in mixture A₂+C+D.

In another preferred embodiment, the step e) of measuring cDNA levels is performed using microarrays.

As used herein, the term “microarray” refers to an arrangement of hybridizable array elements. Preferably, in microarrays used according to the invention, the hybridization signal from each of the array elements is individually distinguishable.

Preferably, when step e) of measuring cDNA levels is performed using microarrays, a relative intensity fluorescence is obtained in step a) for target gene t, and for reference genes g_{c} and g_{d} in each of the at least two cDNA solutions A₁+C+D and A₂+C+D.

In the context of the invention, in particular when step e) of measuring cDNA levels is performed using microarrays and when a relative intensity fluorescence is obtained in step a) for target gene t, and for reference genes g_{c} and g_{d}, any calculation using relative intensity fluorescence values obtained from control gene g_{c} and control gene g_{d} can be sued to normalize the results and give an estimation of experiment's yield between samples A₁ and A₂.

### Kit

The present invention also concerns a kit for comparing the amount of RNA of a target gene t in at least two samples A₁ and A₂, preferably in at least two biological samples A₁ and A₂, comprising:

(i) a determined amount of an external control sample C, preferably of an external control biological sample C, comprising RNA of a reference gene g_{c} and of a reference gene g′_{c};

(ii) a determined amount of external control D RNA including RNA of a reference gene g_{d} and of a reference gene g′_{d};

(iii) a couple of primers that specifically amplify cDNA of the reference gene g_{c};

(iv) a couple of primers that specifically amplify cDNA of the reference gene g′_{c};

(v) a couple of primers that specifically amplify cDNA of the reference gene g_{d}; and

(vi) a couple of primers that specifically amplify cDNA of the reference gene g′_{d};

wherein the reference genes g_{c} and g_{d} are genes with a relative low expression level and the reference genes g′_{c} and g′_{d} are genes with a relative high expression level.

As used herein, the term “couple of primers” refers to oligonucleotides designed to hybridize only to certain regions of target cDNA or external control cDNA to yield amplicons of a specific length in a PCR reaction.

As used herein, the expression “specifically amplify” means that said couple of primers hybridizes to and enables amplifying by PCR a sequence of a given cDNA without hybridizing to or amplifying other sequences.

Preferably, the couple of primers (iii), (iv), (v) and (vi) do not amplify cDNA of the target gene t.

In particular, the reference genes g_{c} and g_{d}, and g′_{c} and g′_{d}, which are specifically amplified by the couple of primers (iii), (iv), (v) and (vi) may be respectively homologous genes obtained from distinct species.

The present invention will be further illustrated, but not limited, by the figures and examples described herein below.

### BRIEF DESCRIPTION OF THE FIGURES

FIG. 1 shows a scheme representing the common and different steps for RT-PCR and microarray experiments.

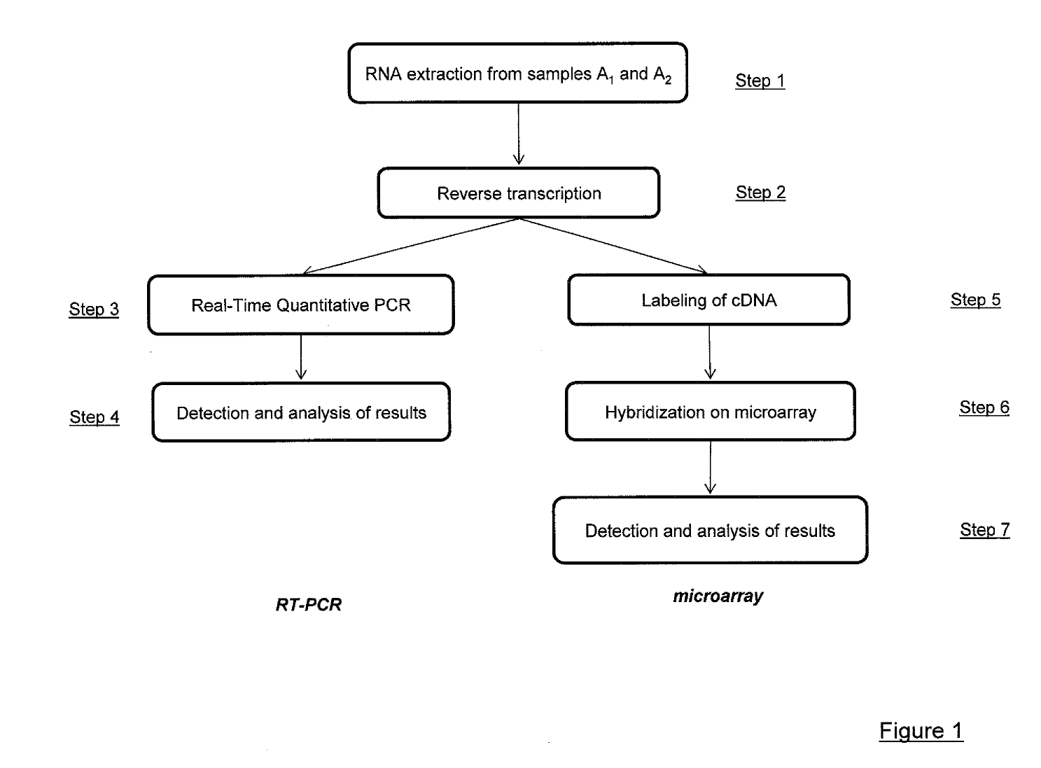

FIG. 2 shows a scheme representing the main steps involved in gene expression analysis between two samples, A₁ and A₂, using quantitative Real-Time PCR using the external controls of the present invention.

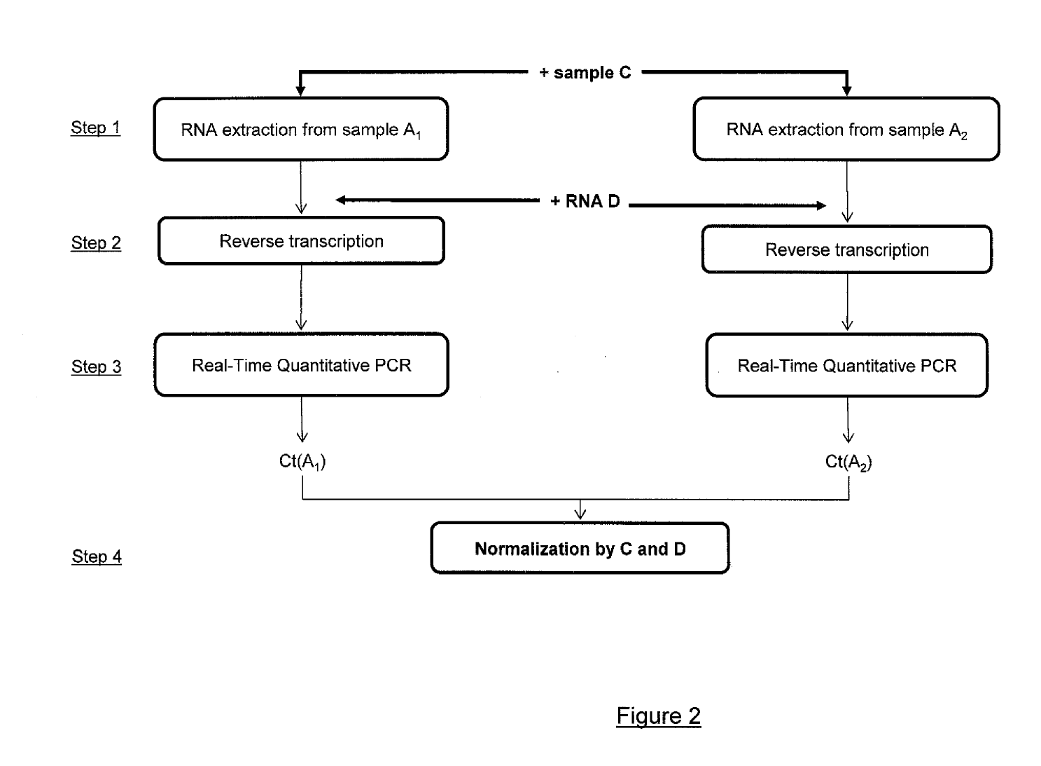

FIG. 3 shows a graph representing Ct variation of 5 HKG (ATP5G3, ACTB, cyclophillin A or PPIA, PGK 1 and Transferrin or TRFC) in normal and cirrhotic liver samples. Corresponding standard deviations and CV values are shown, n=at least 6 for each HKG.

FIG. 4 shows a graph representing Ct variation of 4 HKG (ATP5G3, ACTB, PGK 1 and Transferrin or TRFC) in PNT2 and LNCaP cell lines samples. Corresponding standard deviations and CV values are shown, n=at least 6 for each HKG.

FIG. 5 shows a graph representing the ratio of relative expression of SNAIL in PNT2 and LNCaP cell lines, calculated using Om and Gs external controls or two different HKG genes: ATP5G3 and ACTB. Statistical analyses were performed with Wilcoxon test for intra assay analyses and with Mann & Whitney test for inter assay analyses, with *: 0.05>p>0.01, **: 0.01>p>0.0001, ***: p<0.0001.

FIG. 6 shows a graph representing the ratio of relative expression of SLUG in PNT2 and LNCaP cell lines, calculated using Om and Gs external controls or two different HKG genes: ATP5G3 and ACTB. Statistical analyses were performed with Wilcoxon test for intra assay analyses and with Mann & Whitney test for inter assay analyses, with *: 0.05>p>0.01, **: 0.01>p>0.0001, ***: p<0.0001.

FIG. 7 shows a graph representing the ratio of relative expression of SNAIL in cirrhotic liver tissue and normal liver tissue samples, calculated using Om and Gs external controls or two different HKG genes: ATP5G3 and ACTB. Statistical analyses were performed with Wilcoxon test for intra assay analyses and with Mann & Whitney test for inter assay analyses, with *: 0.05>p>0.01, **: 0.01>p>0.0001, ***: p<0.0001.

FIG. 8 shows a graph representing the ratio of relative expression of SLUG in cirrhotic liver tissue and normal liver tissue samples, calculated using Om and Gs external controls or two different HKG genes: ATP5G3 and ACTB. Statistical analyses were performed with Wilcoxon test for intra assay analyses and with Mann & Whitney test for inter assay analyses, with *: 0.05>p>0.01, **: 0.01>p>0.0001, ***: p<0.0001.

### EXAMPLE

The present example demonstrates the normalizing power of the method according to the invention compared to conventional methods such as methods using HKG.

### Materials and Methods

In the present example, two types of samples A₁ and A₂ of human origin (Homo sapiens, Hs) were studied for their relative expression of SNAIL and SLUG genes (t genes), which are transcription factors involved in mesenchyme-epithelium transition and in cancer process. Samples A₁ and A₂ studied are, on one hand, from normal and cirrhotic liver from two different patients with hepatocarcinoma, and on the other hand, from normal and cancerous prostatic cell lines, i.e. PNT2 and LNCaP respectively.

External control C comes from rainbow trout (Oncorhynchus mykiss, Om) and external control RNA D comes from chicken (Gallus gallus, Gs). GAPDH or ACTS genes from Om and Gs were used in the present example as g_{c}/g_{d} external control genes. The three species (Hs, Om and Gs) do not cross react for target nucleic acid and external control genes chosen for the study.

To ensure the use of standardized amounts of sample from liver biopsy, snap frozen tissues were sectioned using a cryostat-microtome to obtain 50 μm-sections with a diameter of 2 to 3 mm, corresponding to approximately 5 mg of tissue.

The prostatic cell lines PNT2 and LNCaP were maintained at 37° C. in a humidified atmosphere of 5% CO₂, in RPMI 1640 medium (Gibco) supplemented with 10% fetal bovine serum and 1% penistreptomycin-penicillin solution. Cells were harvested at confluence and counted using glasstic slide 10 with grids (KOVA). For RNA extraction, 50,000 cells were pelleted and stored at −80° C.

In the present example, external control C was prepared from rainbow trout muscle (Oncorhynchus mykiss, Om) in the same conditions as human liver samples. Sections of trout tissue were added to the liver samples with a tissue size ratio of 2/1 (sample/trout), and one section of Om muscle was added to 50,000 cells.

Samples and control tissue were sonicated using Bandelin Sonopuls HD 2070 during 30 seconds at 75 W in TRIreagent (Invitrogen) and RNA extraction was done following the manufacturer's instructions.

External control RNA was prepared from chicken muscle (Gallus gallus, Gs) using the TRIreagent (Invitrogen) following manufacturer's instructions.

RNA concentration was measured using Nanodrop spectrophotometer (Thermo Scientific) and 100 ng of control RNA from Gs was added to 400 ng of RNA obtained from mixed samples liver-trout or cells-trout.

Reverse transcription of RNA was performed with M-MLV enzyme (Invitrogen) using random primers following manufacturer's protocol.

cDNAs obtained from RT were used to perform real time rt-qPCR. Real time rt-qPCRs were performed in Step One Plus real-time PCR system (Applied Biosystems) using Sybr®green PCR master mix (Applied Biosystems) according to manufacturer's instructions.

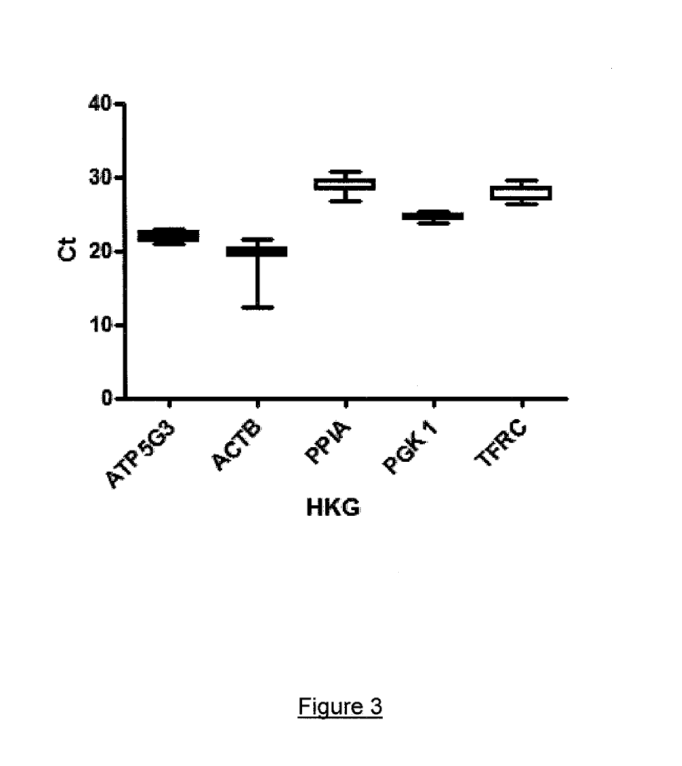

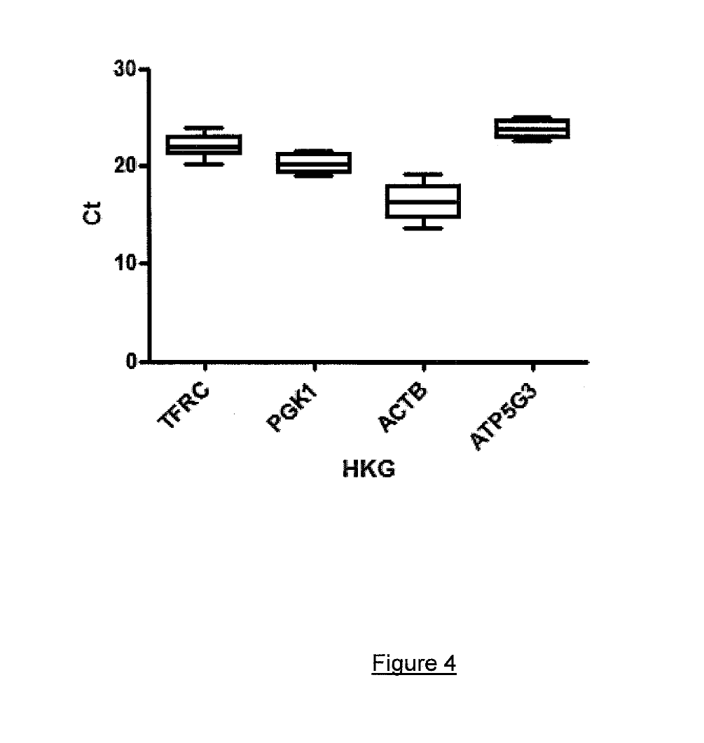

To validate the present invention, results obtained with normalization using external control C and D were compared to results obtained with normalization using the less variable HKG determined beforehand for each experimental condition with statistical analyses (FIGS. 3 and 4). ATP synthase, H⁺ transporting, mitochondrial F0 complex, subunit C3 (ATP5G3) and Phosphoglycerokinase 1 (PGK 1) were subsequently used for normalization of results in prostatic cell lines and liver tissues respectively. Another HKG was tested in cells and liver to evaluate the difference in obtained results and the reproducibility between the best HKG, a less stable HKG compared with the normalization of the present invention.

Expression of target genes, housekeeping genes (HKG) and external control genes was assessed with the following specifically designed forward (F) and reverse (R) primers:

| | |
|---|---|
| 1) | F: 5′-gccttcaactgcaaatactgc-3′ (SEQ ID NO: 1) and R: 5′-tgacatctgagtgggtctgg-3′ |
| | (SEQ ID NO: 2) for Hs SNAIL (SNAIL), |
| 2) | F: 5′-ttcggacccacacattacct-3′ (SEQ ID NO: 3) and R: 5′-ttggagcagtttttgcactg-3′ |
| | (SEQ ID NO: 4) for Hs SLUG (SLUG), |
| 3) | F: 5′-ggatttgccttgtctgaagc-3′ (SEQ ID NO: 5) and R: 5′-cgtacattcccatgacacca-3′ |
| | (SEQ ID NO: 6) for Hs HKG ATP synthase, H+ transporting, mitochondrial FO |
| | complex, subunit C3 (subunit 9) (ATP 5G3), |
| 4) | F: 5′-gaagtggagaaagcctgtgc-3′ (SEQ ID NO: 7) and R: 5′-ctctgtgagcagtgccaaaa-3′ |
| | (SEQ ID NO: 8) for Hs HKG phosphoglycerokinase 1 (PGK 1), |
| 5) | F: 5′-ggggtgttgaaggtctcaaa-3′ (SEQ ID NO: 9) and R: 5′-ggcatcctcaccctgaagta-3′ |
| | (SEQ ID NO: 10) for Hs HKG β-actin (ACTB), |
| 6) | F: 5′-accgtgttcttcgacattgc-3′ (SEQ ID NO: 11) and R: 5′-gcctccacaatattcatgcc-3′ |
| | (SEQ ID NO: 12) for Hs HKG cyclophilin A (PPIA), |
| 7) | F: 5′-GGAGAATCCTGGGGGTTATG-3′ (SEQ ID NO: 13) and R: 5′- |
| | GCTTTCAGCATTTGCAACCT-3′ (SEQ ID NO: 14) for Hs HKG transferrin receptor |
| | (p90, CD71) (TRFC), |
| 8) | F: 5′-gagacaacctggtcctctgtg-3′ (SEQ ID NO: 15) and R: 5′-cttggctggtttctccagac-3′ |
| | (SEQ ID NO: 16) for Gs Glyceraldehyde-3-phosphate dehydrogenase (GAPDH), |
| 9) | F: 5′-cattgagggtctgatgagca-3′ (SEQ ID NO: 17) and R: 5′-aggtccaccactgagacgtt-3′ |
| | (SEQ ID NO: 18) for Om GAPDH, |
| 10) | F: 5′-gactgagaagctgggtttgg-3′ (SEQ ID NO: 19) and R: 5′-tggtaccaccagacagcact-3′ |
| | (SEQ ID NO: 20) for Gs ACTB, |
| 11) | F: 5′-ggcttctctctccaccttcc-3′ (SEQ ID NO: 21) and R: 5′-gactgagaagctgggtttgg-3′ |
| | (SEQ ID NO: 22) for Om ACTB. |

The normalization using housekeeping genes (HKG) was done according to the classical delta Ct equation:

$$R = \frac{{2}^{- [ Ct ( {t}^{{A}_{1}} ) - Ct ( {HKG}^{{A}_{1}} ) ]}}{{2}^{- [ Ct ( {t}^{{A}_{2}} ) - Ct ( {HKG}^{{A}_{2}} ) ]}} \qquad ( Equation 1 )$$

To normalize using external controls, the inventors chose to correct relative expression of SNAIL or SLUG (t genes) in A₁ and A₂ with their respective relative expression of GAPDH or ACTB from Om (g_{c} genes), before correcting sample A₁ with a ratio representing difference in output rt-qPCR between samples A₁ and A₂. This ratio is the comparison of relative expression of GAPDH or ACTB Gs (g_{d} genes) in sample A₂ with those observed in sample A₁. The normalization using external controls was thus done as follows:

$$R = \frac{{2}^{- [ Ct ( {t}^{{A}_{1}} ) - Ct ( {g}_{c}^{{A}_{1}} ) ]}}{{2}^{- [ Ct ( {t}^{{A}_{2}} ) - Ct ( {g}_{c}^{{A}_{2}} ) ]}} \times {2}^{[ Ct ( {g}_{d}^{{A}_{2}} ) - Ct ( {g}_{d}^{{A}_{1}} ) ]} \\ R = \frac{{2}^{- [ Ct ( {t}^{{A}_{1}} ) - Ct ( {g}_{c}^{{A}_{1}} ) ] + Ct ( {g}_{d}^{{A}_{2}} ) - Ct ( {g}_{d}^{{A}_{1}} )}}{{2}^{- [ Ct ( {t}^{{A}_{2}} ) - Ct ( {g}_{c}^{{A}_{2}} ) ]}} \qquad ( Equation 2 )$$

In another way, the difference of real time RT-PCR output between samples A₁ and A₂ can be estimated with direct Ct values, with the coefficient (C) calculated as follows:

$$C = \frac{Ct ( {g}_{d}^{{A}_{2}} )}{Ct ( {g}_{d}^{{A}_{1}} )} \qquad ( Equation 3 )$$

Theoretically, if C is equal or very closed to 1, output difference is negligible and equation 2 can be simplified as follows:

$$R = \frac{{2}^{[ - ( Ct ( {t}^{{A}_{1}} ) - Ct ( {g}_{c}^{{A}_{1}} ) ) \times \frac{Ct ( {g}_{d}^{{A}_{2}} )}{Ct ( {g}_{d}^{{A}_{1}} )} ]}}{{2}^{- [ Ct ( {t}^{{A}_{2}} ) - Ct ( {g}_{c}^{{A}_{1}} ) ]}} \qquad ( Equation 4 )$$

### Results

### Addition of External Controls to Samples

Human samples were assessed with or without external controls (Om and Gs). 500 ng for human samples alone were compared to 400 ng of mixed sample Hs+Om plus 100 ng of Gs RNA. Tables 2 and 3 show the Ct values obtained for SNAIL, SLUG, PGK 1 or ATP5G3 genes in each sample. Corresponding standard deviations and CV values are shown. Unpaired t test was used.

TABLE 2
Ct values for the target genes SNAIL and SLUG and the HKG ATP5G3 obtained in normal liver
tissue and cirrhotic liver tissue in presence or absence of external controls (Om or Gs)

| SNAIL | | SLUG | | PGK 1 | |
|---|---|---|---|---|---|
| Tissue | Tissue + Om + Gs | Tissue | Tissue + Om + Gs | Tissue | Tissue + Om + Gs |

Normal liver

| | | | | | | |
|---|---|---|---|---|---|---|
| Mean Ct | 28.83 | 29.98 | 28.17 | 27.25 | 24.48 | 25.07 |
| ±SD | ±1.44 | ±0.97 | ±1.85 | ±0.99 | ±0.45 | ±0.95 |
| CV | 0.05 | 0.03 | 0.07 | 0.04 | 0.02 | 0.04 |

Cirrhotic liver

| | | | | | | |
|---|---|---|---|---|---|---|
| Mean Ct | 27.34 | 28.88* | 26.13 | 26.22 | 24.61 | 25.37 |
| ±SD | ±2.04 | ±0.70 | ±1.65 | ±0.81 | ±0.52 | ±0.80 |
| CV | 0.07 | 0.02 | 0.06 | 0.03 | 0.02 | 0.03 |

n = at least 15 for each type of sample.

TABLE 3
Ct values for the target genes SNAIL and SLUG and the
HKG ATP5G3 obtained in LNCaP cells and PNT2 cells in
presence or absence of external controls (Om or Gs)

| SNAIL | | SLUG | | ATP5G3 | |
|---|---|---|---|---|---|
| Cells | Cells + Om + Gs | Cells | Cells + Om + Gs | Cells | Cells + Om + Gs |

LNcaP

| | | | | | | |
|---|---|---|---|---|---|---|
| Mean Ct | 28.04 | 26.91** | 29.18 | 27.82*** | 22.83 | 20.97*** |
| ±SD | ±1.47 | ±1.23 | ±1.27 | ±0.72 | ±1.74 | ±0.75 |
| CV | 0.05 | 0.05 | 0.04 | 0.03 | 0.08 | 0.04 |

PNT2

| | | | | | | |
|---|---|---|---|---|---|---|
| Mean Ct | 28.2 | 27.61 | 21.27 | 21.38 | 22.89 | 20.98*** |
| ±SD | ±1.22 | ±1.73 | ±1.07 | ±1.56 | ±1.88 | ±1.18 |
| CV | 0.04 | 0.06 | 0.05 | 0.07 | 0.08 | 0.06 |

n = at least 15 for each type of sample.

Statistical analyses showed that addition of external controls Om and Gs did not significantly alter Ct obtained for each sample studied alone. Even when Hs cell lines were diluted with Om and Gs, rt-qPCR output was better with lower Ct values, above all for LNCaP cells. Since inhibitors of real time RT-PCR could be present in cellular samples, dilution of samples with Om and Gs external controls could decrease inhibitors in samples that could interfere with real time RT-PCR process, leading to better experiment output. Another explanation was the variability in samples preparation between cells alone and cells mixed with Om and Gs. Interestingly, Ct variations of HKG did not systematically follow those of SNAIL and SLUG genes.

### Comparison of Relative Expression Ratios Using HKG(s) or Normalization According to the Present Invention Using External Controls

Ratios of relative expression of SNAIL and SLUG in PNT2 compared to LNCaP were calculated in about 15 different samples of each cell line, following equation 1 using HKG genes expression levels (HKG normalization) and equation 2 using Om and Gs GAPDH expression levels (external control normalization). ATP5G3 was determined as the best HKG, i.e. ATP5G3 showed the less variable expression between PNT2 and LNCaP cells; whereas ACTB was considered the worst HKG tested, i.e. ACTB showed the most variable expression level between PNT2 and LNCaP cells (Table 4 and FIG. 4).

TABLE 4
Ct variation of 4 HKG (ATP5G3, ACTB, PGK 1 and Transferrin or
TRFC) in PNT2 and LNCaP cell lines.

| HKG gene | ATP5G3 | ACTB | PGK 1 | TFRC |
|---|---|---|---|---|
| Mean Ct LNCaP + PNT2 | 23.83 | 16.35 | 20.26 | 22.05 |
| ±SD | ±0.85 | ±1.76 | ±0.92 | ±1.12 |
| CV (%) | 3.6 | 10.8 | 4.6 | 5.1 |

n = at least 6 for each HKG.

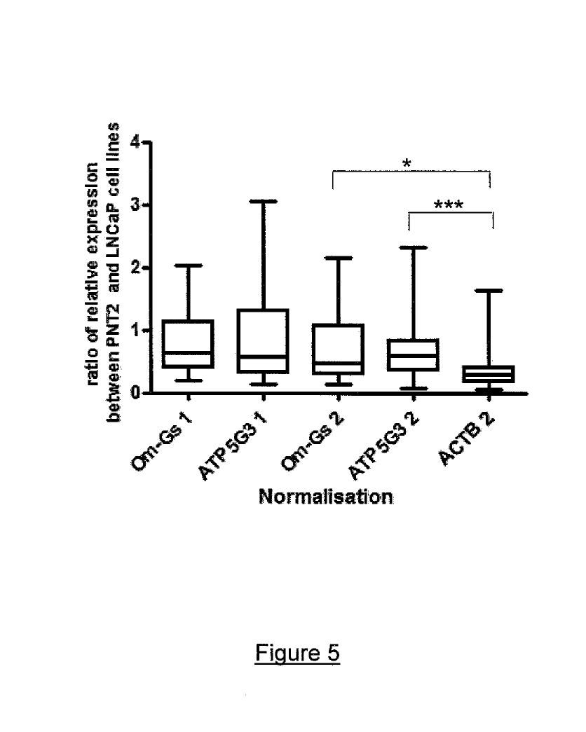

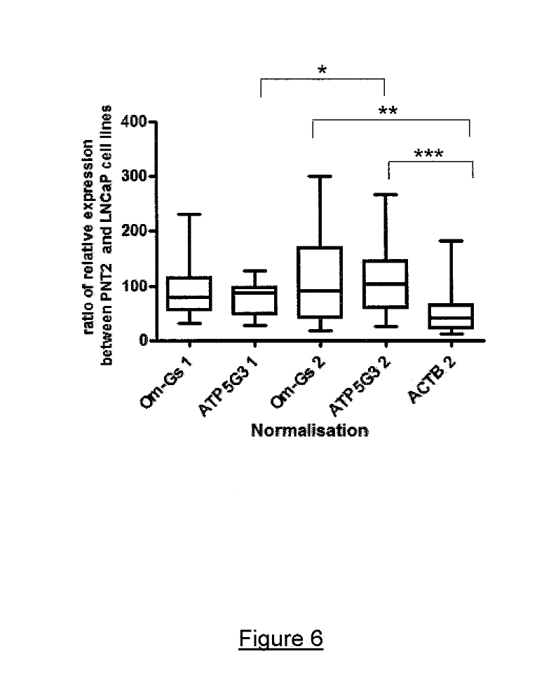

Relative expression of SNAIL and SLUG genes in PNT2 vs LNcaP samples was measured and results from two representative rt-qPCR assays 1 and 2 are depicted in FIGS. 5 and 6 respectively. Concerning SNAIL, no significant difference was observed between normalization with ATP5G3, the best HKG determined, and normalization according to the present invention. In addition, no difference was found between assays 1 and 2 for these two normalizations. However, in assay 2, normalization with ACTB, the less stable HKG determined in the present study, gave ratios of SNAIL between PNT2 and LNCaP two fold lower than normalization with ATP5G3 or normalization using Om and Gs external control. Study of relative expression of SLUG in PNT2 compared to LNCaP cells showed the same results than the study of SNAIL gene, except that normalization with ATP5G3, the best HKG, in assay 1 was significantly different compared to normalization with the same HKG in assay 2. Normalization with Om and Gs external control was similar to the one with ATP5G3, but was the most reproducible (Table 5), showing better coefficient of variation.

TABLE 5
Inter-assay reproducibility for ratios values of SNAIL and SLUG genes.

| | |
|---|---|
| Om-Gs | ATP5G3 |

SNAIL

| | | |
|---|---|---|
| Mean | 0.75 | 0.80 |
| ±SD | ±0.04 | ±0.06 |
| CV | 0.05 | 0.08 |

SLUG

| | | |
|---|---|---|
| Mean | 101.70 | 93.96 |
| ±SD | ±8.12 | ±17.75 |
| CV | 0.08 | 0.19 |

Ratios of relative expression of SNAIL and SLUG in cirrhotic compared to normal liver were calculated in about 20 different samples of each tissue, following equation 1 using HKG genes expression levels (HKG normalization) and equation 2 using Om and Gs ACTB expression levels (external control normalization). PGK 1 was determined as the less variable HKG, but ATP5G3 was also a quite good candidate for a stable HKG (FIG. 3 and Table 6), so normalization with ATP5G3 was studied in parallel for comparison with PGK 1 normalization and normalization using Om and Gs external control described in the present invention.

TABLE 6
Ct variation of 5 HKG (ATP5G3, ACTB, cvclophillin A or PPIA, PGK 1
and Transferrin or TRFC) in normal and cirrhotic liver samples.

| HKG gene | ATP5G3 | ACTB | PPIA | PGK 1 | TFRC |
|---|---|---|---|---|---|
| Mean Ct NL + CL | 21.93 | 19.63 | 28.78 | 24.54 | 27.70 |
| ±SD | ±0.57 | ±2.00 | ±1.12 | ±0.46 | ±0.95 |
| CV (%) | 2.6 | 10.2 | 3.9 | 1.9 | 3.4 |

n = at least 6 for each HKG.

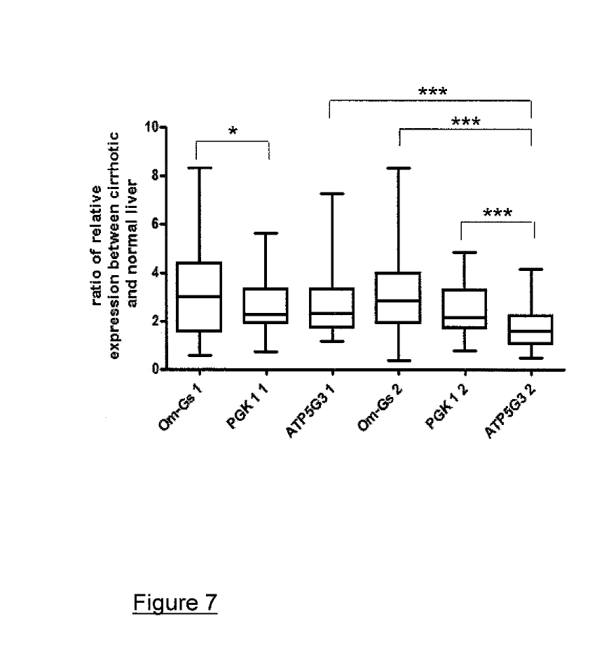

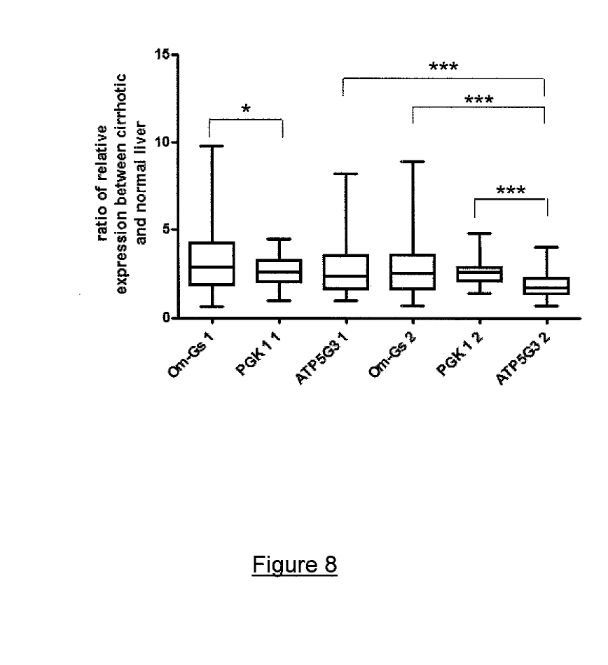

Relative expression of SNAIL and SLUG genes in cirrhotic liver vs normal liver tissues was measured and results from two representative real time RT-PCR assays 1 and 2 are depicted in FIGS. 7 and 8 respectively. For both SNAIL and SLUG relative expression studied, ratios calculated with PKG 1 normalization and with Om and Gs external control normalization was similar between assays 1 and 2. In assay 1, normalization using PGK 1 and external control gene was significantly different. This could be due to higher variations in ratios obtained with normalization using external control gene. The inventors have already observed a variation in HKG/gene expression according to the localization of biopsy in the organ. This could explain the higher variation of ratios calculated with external control genes which expression does not vary according to the localization of the biopsy. In assay 1, normalization with ATP5G3 seemed to be similar to normalization using PGK 1 or Om and Gs external control genes. However in assay 2, normalization with ATP5G3 was significantly different from ATP5G3 normalization in assay 1 and from PGK 1 and Om-Gs normalizations in assay 2. Even if ATP5G3 seemed to be a quite good candidate as a stable HKG, its use in normalization did not give reproducible results. Finally, normalization using PGK 1 and Om and Gs external control gave comparable coefficient of variation when estimated inter assay reproducibility of the technique was assessed (Table 7).

TABLE 7
Inter-assay reproducibility for ratio values of SNAIL and SLUG genes.

| | | |
|---|---|---|
| Om-Gs | PGK | ATP5G3 |

SNAIL

| | | | |
|---|---|---|---|
| Mean | 3.20 | 2.54 | 2.26 |
| ±SD | ±0.057 | ±0.06 | ±0.54 |
| CV | 0.02 | 0.02 | 0.24 |

SLUG

| | | | |
|---|---|---|---|
| Mean | 3.18 | 2.66 | 2.41 |
| ±SD | ±0.09 | ±0.03 | ±0.51 |
| CV | 0.03 | 0.01 | 0.21 |

All these data demonstrated that normalization using Om and Gs external controls did not interfere with the different steps of real time RT-PCR and that relative expression levels obtained were as reliable as classical HKG normalization, provided that HKG were determined in pre-analytical experiments as the less variable in samples studied.

### Advantages of the Use of External Controls of the Present Invention

Normalization using Om and Gs external controls, described in the present example, gave similar results to those obtained with normalization using the less variable HKG, determined with pre-analytical experiments. In addition, normalization with Om and Gs external controls was reproducible. These results demonstrated that normalization according to the present invention is as efficient as the best HKG determined. The invention could be used in replacement of HKG, avoiding time-, money- and sample-consuming pre-analytical experiments needed to determine the less variable HKG.

In addition, variations of HKG expression could be observed within the same tissue, according to the localization of the biopsy. This variation could interfere with a good normalization of real time RT-PCR results. Om and Gs external control genes have stable expression, whatever the sample or the localization of the biopsy studied.

Moreover, external controls were essential to pinpoint problems encountered during extraction and/or RT and PCR steps. Indeed, in Tables 8 and 9, cell line or tissues liver samples with high Ct values for Om and Gs are shown.

TABLE 8
Aberrant Ct values for cell line samples not detected with HKG normalization.

| | Ct | | | | | Ratio PNT2/LNCaP | |
|---|---|---|---|---|---|---|---|
| | SNAIL | SLUG | ATP5G3 | GAPDH Om | GAPDH Gs | SNAIL | SLUG |
| Aberrant | 27.71 | 35.50 | 24.38 | 35.02 | 35.88 | 0.12 | 1741.82 |
| LNCaP | 30.97 | 36.14 | 25.66 | 36.24 | Undetermined | 0.47 | 1124.58 |
| samples | 31.65 | 34.19 | 26.17 | 35.56 | 36.17 | 0.53 | 203.97 |
| | 29.97 | 35.83 | 24.29 | Undetermined | 35.52 | 0.60 | 2342.38 |
| Theoretical | 26.89 | 27.82 | 20.98 | 20.12 | 16.06 | 0.71 | 89.99 |
| LNCaP | | | | | | | |
| sample | | | | | | | |
| (mean) | | | | | | | |
| Aberrant | 24.96 | 35.14 | 22.75 | 3.51 | 32.63 | 13.06 | 0.02 |
| PNT2 | 30.43 | 35.76 | 25.98 | 35.82 | 33.91 | 2.76 | 0.13 |
| samples | | | | | | | |
| Theoretical | 27.46 | 21.42 | 21.06 | 19.93 | 16.90 | | |
| PNT2 | | | | | | | |
| sample | | | | | | | |
| (mean) | | | | | | | |

Ratios were calculated using HKG for each aberrant Ct values for SNAIL and SLUG genes using Ct from aberrant samples A₁ or A₂ and mean Ct of the counterpart sample A₂ or A₁. For comparison, theoretical ratio using mean Ct of samples A₁ and A₂ were calculated.

TABLE 9
Aberrant Ct values for tissue samples not detected with HKG normalization.

| | Ct | | | | | Ratio CL/NL | |
|---|---|---|---|---|---|---|---|
| | SNAIL | SLUG | ATP5G3 | GAPDH Om | GAPDH Gs | SNAIL | SLUG |
| Aberrant | 31.50 | 29.01 | 26.84 | 30.03 | 23.13 | 2.21 | 2.50 |
| normal liver | | | | | | | |
| samples | | | | | | | |
| Theoretical | 29.98 | 27.25 | 25.07 | 27.52 | 22.09 | 2.64 | 2.53 |
| normal liver | | | | | | | |
| sample (mean) | | | | | | | |
| Aberrant | 31.15 | 28.79 | 27.52 | 32.13 | 23.52 | 2.43 | 1.88 |
| cirrhotic liver | 33.62 | 28.93 | 28.27 | 32.52 | 25.13 | 0.74 | 2.88 |
| samples | 36 | 31.15 | 28.79 | 27.52 | 32.13 | 0.20 | 0.88 |
| | 31.50 | 29.01 | 26.84 | 30.03 | 23.13 | 1.19 | 1.01 |
| Theoretical | 28.88 | 26.22 | 25.37 | 28.11 | 22.21 | | |
| cirrhotic liver | | | | | | | |
| sample (mean) | | | | | | | |

Ratios were calculated using HKG for each aberrant Ct values for SNAIL and SLUG genes using Ct from aberrant samples A₁ or A₂ and mean Ct of the counterpart samples A₂ or A₁. For comparison, theoretical ratio using mean Ct of sample A₁ and A₂ were calculated.

High Ct values for Om and Gs were most probably due to problems during processing of the samples. In the same samples, HKG ATP5G3 or PGK 1 Ct values were higher but within normal range of gene expression. Thus, when using HKG for data normalization, target gene expression could not be correctly observed, whereas using Om and Gs external controls, problems in output of real time RT-PCR could be revealed. These results show that HKG normalization could lead to a misinterpretation because it does not reflect variation due to sample processing.

Finally, the present invention can be commercialized to propose a worldwide standardization of real time rt-qPCR and microarray results, to enable inter-laboratories genomic expression comparison. External control genes can be chosen according to their relative expression and to the expression of the gene of interest in samples A₁ and A₂. Indeed, external control genes with low expression could be used to study target genes with a low expression in samples of interest, inversely external control genes with high expression could be used to study highly expressed genes of interest. This method offers the possibility to normalize results with control genes and target genes showing close Ct values, which is not possible with HKG since they are generally highly expressed.

1. A method for comparing, in at least two samples A₁ and A₂, the amount of RNA of a target gene t, comprising the steps consisting of:
a) mixing each of the at least two samples A₁ and A₂ with a determined amount of external control sample C comprising RNA of a reference gene g_{c};
b) extracting RNA from each of the at least two mixtures A₁+C and A₂+C obtained in step a), in order to obtain corresponding solutions of extracted RNA;
c) mixing each of the at least two solutions of extracted RNA of A₁+C and A₂+C with a determined amount of external control D RNA including RNA of a reference gene g_{d};
d) performing reverse transcriptions on each of the at least two mixtures A₁+C+D and A₂+C+D obtained in step c), in order to obtain corresponding solutions comprising cDNAs of the target gene t, of the reference gene g_{c} and of the reference gene g_{d};
e) measuring the cDNA levels of each of the target gene t, of the reference gene g_{c} and of the reference gene g_{d} in each of the at least two cDNA solutions A₁+C+D and A₂+C+D obtained in step d); and
f) normalizing the cDNA levels of the target gene t from the at least two samples A₁ and A₂, using cDNA levels of the reference genes g_{c} and g_{d};
wherein the reference gene g_{c} is selected in such a way that nucleic acids, primers and/or probes used in step e) to measure the cDNA level of the reference gene g_{c} do not cross-react with cDNAs of the target gene t and of the reference gene g_{d} and wherein the reference gene g_{d} is selected in such a way that nucleic acids, primers and/or probes used in step e) to measure the cDNA level of the reference gene g_{d} do not cross-react with cDNAs of the target gene t and of the reference gene g_{c}.

2. The method according to claim 1, wherein the reference genes g_{c} and g_{d} are selected in such a way that the order of magnitude of their relative expression level is similar to the order of magnitude of the relative expression level of the target gene t.

3. The method according to claim 1, wherein step e) of measuring cDNA levels is performed by quantitative PCR.

4. The method according to claim 3, wherein a cycle threshold Ct value is obtained in step e) for target gene t, and for reference genes g_{c} and g_{d} in each of the at least two cDNA solutions A₁+C+D and A₂+C+D.

5. The method according to claim 4, wherein step f) of normalizing is performed using the following equation:
$$R = \frac{{2}^{- [ Ct ( {t}^{{A}_{1}} ) - Ct ( {g}_{c}^{{A}_{1}} ) ] + Ct ( {g}_{d}^{{A}_{2}} ) - Ct ( {g}_{d}^{{A}_{1}} )}}{{2}^{- [ Ct ( {t}^{{A}_{2}} ) - Ct ( {g}_{c}^{{A}_{2}} ) ]}} ;$$
wherein:
R represents the ratio of the cDNA level of the target gene t in the sample A₁ on the cDNA level of the target gene t in the sample A₂;
Ct(t^{A}^{₁}) represents the cycle threshold obtained for the target gene t in the mixture A₁+C+D;
Ct(t^{A}^{₂}) represents the cycle threshold obtained for the target gene t in the mixture A₂+C+D;
Ct(g_{c}^{A}^{₁}) represents the cycle threshold obtained for the reference gene g_{c} in the mixture A₁+C+D;
Ct(g_{c}^{A}^{₂}) represents the cycle threshold obtained for the reference gene g_{c} in mixture A₂+C+D;
Ct(g_{d}^{A}^{₁}) represents the cycle threshold obtained for the reference gene g_{d} in the mixture A₁+C+D; and
Ct(g_{d}^{A}^{₂}) represents the cycle threshold obtained for the reference gene g_{d} in mixture A₂+C+D.

6. The method according to claim 1, wherein the step e) of measuring cDNA levels is performed using microarrays.

7. The method according to claim 6, wherein a relative intensity fluorescence is obtained in step a) for target gene t, and for reference genes g_{c} and g_{d} in each of the at least two cDNA solutions A₁+C+D and A₂+C+D.

8. A kit for comparing the amount of RNA of a target gene t in at least two samples A₁ and A₂, comprising:
(i) a determined amount of an external control sample C comprising RNA of a reference gene g_{c} and of a reference gene g′_{c};
(ii) a determined amount of external control D RNA including RNA of a reference gene g_{d} and of a reference gene g′_{d};
(iii) a couple of primers that specifically amplify cDNA of the reference gene g_{c};
(iv) a couple of primers that specifically amplify cDNA of the reference gene g′_{c};
(v) a couple of primers that specifically amplify cDNA of the reference gene g_{d}; and
(vi) a couple of primers that specifically amplify cDNA of the reference gene g′_{d};
wherein the reference genes g_{c} and g_{d} are genes with a relative low expression level and the reference genes g′_{c} and g′_{d} are genes with a relative high expression level.

9. The kit according to claim 8, wherein the couple of primers (iii), (iv), (v) and (vi) do not amplify cDNA of the target gene t.

10. The method according to claim 2, wherein step e) of measuring cDNA levels is performed by quantitative PCR.

11. The method according to claim 2, wherein the step e) of measuring cDNA levels is performed using microarrays.
